(19) United States
(12) Patent Application Publication
(10) Pub. No.: US 2015/0064110 A1
(43) Pub. Date: Mar. 5, 2015

(54) CROSSLINKED POLYSACCHARIDE BEADS COMPRISING AN IMAGING AGENT

(71) Applicants: INSERM (Institut National de la Sante et de la Recherche Medicale), Paris (FR); Universite Paris Diderot - Paris 7, Paris (FR); Univ Paris XIII Paris-Nord Villetaneuse, Villetaneuse (FR)

(72) Inventors: Sidi Mohammed Derkaoui, Paris (FR); Catherine Le Visage, Paris (FR); Thomas Bonnard, Paris (FR); Didier LeTourneur, Paris (FR); Jean-Michel Serfaty, Nantes cedex 1 (FR)

(73) Assignees: INSERM (Institut National de la Sante et de la Recherche Medicale), Paris (FR); Universite Paris Diderot - Paris 7, Paris (FR); Univ Paris XIII Paris-Nord Villetaneuse, Villetaneuse (FR)

(21) Appl. No.: 14/379,846

(22) PCT Filed: Feb. 22, 2013
(86) PCT No.: PCT/EP2013/053608

§ 371 Date: Aug. 20, 2014

(30) Foreign Application Priority Data
Feb. 24, 2012 (EP) 12305224.3

(51) Int. Cl. A61K 47/36; A61K 49/18; A61K 49/22; A61K 51/12; A61K 51/06

(52) U.S. Cl. CPC A61K 47/36; A61K 51/1251; A61K 51/06; A61K 49/225; A61K 49/1887; A61K 49/1824
USPC 424/1.65; 424/9.32; 424/9.5

(57) ABSTRACT

The present invention relates to a method for preparing beads comprising imaging agent. The present invention further provides beads highly useful for medical imaging.

### FIELD OF THE INVENTION

The present invention relates to a method for preparing beads comprising imaging agent. The present invention further provides beads highly useful for medical imaging.

### BACKGROUND OF THE INVENTION

Medical imaging is a widely used technique allowing the visualization of the organs and the cells within a human body. For several critical conditions medical imaging became a key factor in the diagnosis of said conditions and the management of the appropriate therapies.

New strategies for carrying out such imaging are currently under investigation, especially as for the development of new imaging molecules. Proper choice of an imaging molecule constitutes the key for providing an accurate image.

Imaging molecules need to provide very target-specific binding and sufficient stability in the circulation to allow strong and selective accumulation. They need to be non-toxic and sensitively detectable. Highly controlled physical characteristics should help obtain more uniform kinetic behavior and contribute to the implementation of quantitative detection techniques.

Because of problems inherent with the use of many presently available imaging molecules, there is an unfulfilled need for new agents adaptable for clinical use.

There is indeed a need for imaging molecules which alleviates concerns with known imaging molecules and allows high contrast images to be achieved, with low toxicity. It would also be desirable to provide molecules for use in medical imaging that provide specific biodistribution (permitting a variety of organs to be targeted), have a size sufficiently small to permit free circulation through a subject's vascular system or by blood perfusion, while being ultimately metabolized and/or excreted by the subject. There is also a need for drug delivery materials that allow drugs or other therapeutic agents to be delivered to tissues or portions of the body in an effective manner. There is also a need for agents that allow drugs or other therapeutic agents to be introduced into cells of the body.

### SUMMARY OF THE INVENTION

The inventors fill the foregoing need by new molecules and strategies for carrying out an imaging method. The inventors indeed developed beads, useful for several well known imaging techniques such as MRI, ultrasound and scintigraphy. More specifically, the inventors developed a method for preparing beads comprising an imaging agent. Said method is advantageous since it may easily be adapted for providing a very specific type of beads, depending on its purpose and adapted to a given imaging technique. In addition, the inventors developed a method for obtaining a new kind of entity for imaging, which are polysaccharide beads comprising an imaging agent and which may be specific to selectins thanks to the presence of fucoidan. The inventors therefore met the burden to create a new class of entities useful for imaging techniques. In addition, they further developed a new class of molecules able to target a specific tissue while imaging.

In a first object, the invention relates to a method for preparing beads comprising an imaging agent comprising the following steps:
i) preparing an alkaline aqueous solution comprising an amount of at least one polysaccharide, an amount of an imaging agent and an amount of a cross linking agent;
ii) dispersing said alkaline aqueous solution into an hydrophobic phase comprising a surfactant in order to obtain w/o emulsion; and
iii) transforming the w/o emulsion into beads by placing said w/o emulsion at a temperature from about 4° C. to about 80° C. for a sufficient time to allow the cross-linking of said amount of polysaccharide;
   wherein,
   said polysaccharide is selected from the group consisting of dextran, pullulan, fucoidan, agar, alginic acid, hyaluronic acid, inulin, heparin, chitosan and mixtures thereof.

In one embodiment, said polysaccharide is fucoidan. In another embodiment, said polysaccharide is pullulan.

In another embodiment, the method of the invention comprises a further step iv) of incubating the beads obtained in step iii) with a solution of fucoidan to graft fucoidan on the surface of the beads.

In a second aspect, the invention provides a method for preparing beads comprising radioactive imaging compounds comprising the following steps:
a) preparing an alkaline aqueous solution comprising an amount of fucoidan, an amount of at least one polysaccharide and an amount of a cross linking agent;
b) dispersing said alkaline aqueous solution into an hydrophobic phase comprising a surfactant in order to obtain w/o emulsion;
c) transforming the w/o emulsion into beads by placing said w/o emulsion at a temperature from about 4° C. to about 80° C. for a sufficient time to allow the cross-linking of said amount of polysaccharide; and
d) putting the obtained beads in contact with radioactive imaging compounds chosen in the group consisting of carbon-11 (¹¹C), nitrogen-13 (¹³N), oxygen-15 (¹⁵O) and fluorine-18 (¹⁸F), gallium-68 (⁶⁸Ga), yttrium-91 (⁹¹Y), indium-111 (¹¹¹In), rhenium-186 (¹⁸⁶Re), thallium-201 (²⁰¹Tl), terbium and mixtures thereof:
   wherein,
   said polysaccharide is selected from the group consisting of dextran, pullulan, fucoidan, agar, alginic acid, hyaluronic acid, inulin, heparin, chitosan and mixtures thereof.

In one embodiment, said polysaccharide is fucoidan. In another embodiment, said polysaccharide is pullulan.

In another embodiment, the method of the invention comprises a step c′) after step c) and before step d) of incubating the beads obtained in step c) with a solution of fucoidan to graft fucoidan on the surface of the beads.

In a third aspect, the invention relates to a bead comprising an imaging agent obtainable by any of the methods of the invention.

In a fourth aspect, the invention relates to the use of the bead obtainable by any of the methods of the invention as a contrast agent.

### DETAILED DESCRIPTION OF THE INVENTION

Definition

As used herein, the term “polysaccharide” refers to a molecule comprising two or more monosaccharide units.

As used herein, the term “alkaline solution” refers to a solution having a pH strictly superior to 7.

As used herein, the term “aqueous solution” refers to a solution in which the solvent is water.

As used herein, the term “cross-linking” refers to the linking of one polysaccharide chain to another one with covalent bonds.

As used herein, the term “cross-linking agent” encompasses any agent able to introduce cross-links between the chains of the polysaccharides of the invention.

As used herein, the term “beads” is used in an interchangeable manner and refer to polysaccharide composition obtainable according to any of the methods of the invention and having a substantially spherical or ovoid shape.

As used herein, the expression “beads of the invention”, “imaging entities” or “contrasting agent” are used interchangeably refer to the beads obtained by the methods of the invention, i.e. beads comprising an imaging agent.

As used herein, the term “nanobeads” encompasses bead shaving a size of at least 1 nm and inferior to 1000 nm, the term “microbeads” encompasses beads having a size of at least 1 μm and inferior to 1000 μm, the term “macrobeads” encompasses beads having a size of at least to 1 mm.

As used herein, the term “biodegradable” refers to materials that degrade in vivo to non-toxic compounds, which can be excreted or further metabolized.

As used herein, the term “freeze-drying” refers to the drying of a deep-frozen material under high vacuum by freezing out the solvent (ie. water) and then evaporating it in the frozen state.

As used herein, the term “surfactant” refers to a compound that lowers the surface tension of water.

As used herein, the terms “non-aqueous phase”, “lipophilic phase”, “hydrophobic phase”, and “oily phase” may be used in an interchangeable manner.

As used herein, “w/o emulsion” or “water-in-oil emulsion”, refers to the dispersion of an aqueous phase into a lipophilic phase. The term “w/o emulsion” encompasses stable and non-stable emulsion.

As used herein, the term “imaging agent” refers to a molecule that can be used to detect specific biological elements using imaging techniques. Therefore, the term encompasses molecules detectable by well known imaging techniques such as planar scintigraphy (PS), Single Photon Emission Computed Tomography (SPECT), Positron Emission Tomography (PET), contrast-enhanced ultrasonography (CEUS), Magnetic Resonance Imaging (MRI), fluorescence spectroscopy, Computed Tomography, ultrasonography, X-ray radiography, or any combination thereof.

In the context of the invention, said term encompasses:
A) MRI imaging compounds selected in the group consisting of ultrasmall uperparamagnetic iron oxide particles (USPIOs), gadolinium III (Gd³⁺), chromium III (Cr³⁺), dysprosium III (Dy³⁺), iron III (Fe³⁺), manganese II (Mn²⁺), and ytterbium III (Yb³⁺), and mixtures thereof;
B) radioactive imaging compounds selected in the group consisting of carbon-11 (¹¹C), nitrogen-13 (¹³N), oxygen-15 (¹⁵O) and fluorine-18 (¹⁸F), gallium-68 (⁶⁸Ga), yttrium-91 (⁹¹Y), technetium-99m (^{99m}Tc), indium-111 (¹¹¹In), iodine-131 (¹³¹I), rhenium-186 (¹⁸⁶Re), and thallium-201 (²⁰¹Tl), terbium and mixtures thereof;
C) contrast-enhanced ultrasonography imaging compounds such as perfluoroctyl bromide (PFOB);
D) fluorescence imaging compounds selected in the group consisting of quantum dots (i.e., fluorescent inorganic semiconductor nanocrystals) and fluorescent dyes such as Texas red, fluorescein isothiocyanate (FITC), phycoerythrin (PE), rhodamine, fluorescein, carbocyanine, Cy-3™ and Cy-5™ (i.e., 3- and 5-N,N'-diethyltetra-methylindodicarbocyanine, respectively), Cy5.5, Cy7, DY-630, DY-635, DY-680, and Atto 565 dyes, merocyanine, styryl dye, oxonol dye, BODIPY dye (i.e., boron dipyrromethene difluoride fluorophore), terbium, and
E) mixtures thereof.

As used herein, the expression “beads comprising fucoidan” refers to beads comprising fucoidan obtainable according to the methods of the invention. In one embodiment, said beads are obtained directly by the methods of the invention because of the presence of fucoidan in the alkaline aqueous solution of step i) or a). In this particular embodiment, the fucoidan is thus in the very structure of the bead. Its presence can be found within the bead as well as on the surface of said beads. In an alternative embodiment, said beads are obtained by incubating the beads of the invention with a solution of fucoidan to graft fucoidan on the surface of the beads. In this particular embodiment, the fucoidan is thus on the surface of the beads.

The term “fucoidan” or “fucoidan moiety” refers to any fucoidan entity exhibiting high affinity, specificity and/or selectivity for selectins. As used herein, the term “selectin” has its art understood meaning and refers to any member of the family of carbohydrate-binding, calcium-dependent cell adhesion molecules that are constitutively or inductively present on the surface of leukocytes, endothelial cells or platelets. The term “E-selectin”, as used herein, has its art understood meaning and refers to the cell adhesion molecule also known as SELE, CD62E, ELAM, ELAM1, ESEL, or LECAM2 (Genbank Accession Numbers for human E-selectin: NM_{—}000450 (mRNA) and NP_{—}000441 (protein)). As used herein, the term “L-selectin” has its art understood meaning and refers to the cell adhesion molecule also known as SELL, CD62L, LAM-1, LAM1, LECAM1, LNHR, LSEL, LYAM1, Leu-8, Lyam-1, PLNHR, TQ1, or hLHRc (Genbank Accession Numbers for human L-selectin: NM_{—}000655 (mRNA) and NP_{—}000646 (protein)). The term “P-selectin”, as used herein, has its art understood meaning and refers to the cell adhesion molecule also known as a SELP, CD62, CD62P, FLJ45155, GMP140, GRMP, PADGEM, or PSEL (Genbank Accession Numbers for human P-selectin: NM_{—}003005 (mRNA) and NP_{—}002996 (protein)).

The terms “binding affinity” and “affinity” are used herein interchangeably and refer to the level of attraction between molecular entities. Affinities can be expressed quantitatively as a dissociation constant (K_{d} or K_{D}), or its inverse, the association constant (Kₐ or K_{A}).

The terms “pathological condition associated with selectins”, “disease associated with selectins” and “disorder associated with selectins” are used herein interchangeably. They refer to any disease condition characterized by undesirable or abnormal selectin-mediated interactions. Such conditions include, for example, disease conditions associated with or resulting from the homing of leukocytes to sites of inflammation, the normal homing of lymphocytes to secondary lymph organs, the interaction of platelets with activated endothelium, platelet-platelet and platelet-leukocyte interactions in the blood vascular compartment, and the like. Examples of such disease conditions include, but are not limited to, tissue transplant rejection, platelet-mediated diseases (e.g., atherosclerosis and clotting), hyperactive coronary circulation, acute leukocyte-mediated lung injury (e.g., adult respiratory distress syndrome—ARDS), Crohn's disease, inflammatory diseases (e.g., inflammatory bowel disease), autoimmune diseases (e.g., multiple sclerosis, myasthenia gravis), infection, cancer (including metastasis), thrombosis, wounds and wound-associated sepsis, burns, spinal cord damage, digestive tract mucous membrane disorders (e.g., gastritis, ulcers), osteoporosis, rheumatoid arthritis, osteoarthritis, asthma, allergy, psoriasis, septic shock, stroke, nephritis, atopic dermatitis, frostbite injury, adult dysponoea syndrome, ulcerative colitis, systemic lupus erythrematosis, diabetes and reperfusion injury following ischemic episodes.

As used herein, the term “subject” refers to a human or another mammal (e.g., mouse, rat, rabbit, hamster, dog, cat, cattle, swine, sheep, horse or primate). In many embodiments, the subject is a human being. In such embodiments, the subject is often referred to as an “individual” or to a “patient” if the subject is afflicted with a disease or clinical condition. The terms “subject”, “individual” and “patient” do not denote a particular age, and thus encompass adults, children and newborns.

The term “effective amount”, when used herein in reference to an imaging agent refers to any amount of said imaging agent which is sufficient to fulfill its intended purpose(s) (e.g., the purpose may be the detection and/or imaging of selectins present in a biological system or in a subject, and/or the diagnosis of a disease associated with selectins).

The term “biological sample” is used herein in its broadest sense. A biological sample is generally obtained from a subject. A sample may be of any biological tissue or fluid that can produce and/or contain selectins. Frequently, a sample will be a “clinical sample”, i.e., a sample derived from a patient. Such samples include, but are not limited to, bodily fluids which may or may not contain cells, e.g., blood, urine, saliva, cerebrospinal fluid (CSF), cynovial fluid, tissue or fine needle biopsy samples, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample, proteins or other molecules extracted from the sample. Processing of a biological sample may involve one or more of: filtration, distillation, extraction, concentration, inactivation of interfering components, addition of reagents, and the like.

As used herein, the abbreviation “MPIO” refers to beads of the invention comprising at least one USPIO.

Asu used herein, the abbreviation “MPIO-Fucoidan” refers to beads of the invention comprising at fucoidan and USPIO.

As used herein, the abbreviation “MP-PFOB” refers to beads of the invention comprising at least one PFOB molecule.

As used herein, the abbreviation “MP-PFOB-Fucoidan” refers to beads of the invention comprising fucoidan and PFOB.

As used herein, the abbreviation “MP-^{99m}Tc” refers to beads of the invention comprising at least one ^{99m}Technetium molecule. The abbreviation “MP-^{99m}Tc-fucoidan” refers to microparticles of the invention comprising at least one fucoidan and ^{99m}Technetium.

Method for Preparing Crosslinked Beads Comprising an Imaging Agent

In a first object, the invention relates to a method for preparing beads comprising an imaging agent comprising the following steps:
i) preparing an alkaline aqueous solution comprising an amount of at least one polysaccharide, an amount of at least one imaging agent and an amount of a cross linking agent;
ii) dispersing said alkaline aqueous solution into an hydrophobic phase comprising a surfactant in order to obtain w/o emulsion; and
iii) transforming the w/o emulsion into beads by placing said w/o emulsion at a temperature from about 4° C. to about 80° C. for a sufficient time to allow the cross-linking of said amount of polysaccharide;
   wherein
   said polysaccharide is selected from the group consisting of dextran, pullulan, fucoidan, agar, alginic acid, hyaluronic acid, inulin, heparin, chitosan and mixtures thereof.

By encapsulating the imaging agent in the polysaccharide beads, the invention provides improved molecules useful for imaging techniques. Indeed, the beads of the invention may concentrate a large amount of imaging agents, which ultimately brings a much higher density of imaging agents at the site to be imaged, and therefore a much better contrast. In this embodiment, the imaging agent is encapsulated within the structure of the bead.

In a specific embodiment said polysaccharide is fucoidan. Step iii) therefore provide a bead comprising fucoidan and an imaging agent.

Alternatively, the method of the invention further comprises a step iv) of incubating the beads obtained in step iii) with a solution of fucoidan to graft fucoidan on the surface of the beads. In this embodiment, step iv) thus provides:
a polysaccharide bead comprising an imaging agent with fucoidan on its surface; or
a polysaccharide bead comprising imaging agent having fucoidan on its surface, wherein said polysaccharide is fucoidan.

In both of these embodiments, by coupling an imaging agent to a bead comprising fucoidan, the invention provides a new class of imaging entities which are useful in several medical imaging techniques such as MRI signal, in ultrasound or in scintigraphy and in which the imaging entities specifically target a given tissue, thanks to the selectivity of fucoidan toward selectins.

Preferably, said at least one imaging agent is chosen among:
A) MRI imaging compounds selected in the group consisting of ultrasmall superparamagnetic iron oxide particles (USPIOs), gadolinium III (Gd³⁺), chromium III (Cr³⁺), dysprosium III (Dy³⁺), iron III (Fe³⁺), manganese II (Mn²⁺), and ytterbium III (Yb³⁺), and mixtures thereof;
B) radioactive imaging compounds selected in the group consisting of carbon-11 (¹¹C), nitrogen-13 (¹³N), oxygen-15 (¹⁵O) and fluorine-18 (¹⁸F), gallium-68 (⁶⁸Ga), yttrium-91 (⁹¹Y), technetium-99m (^{99m}Tc), indium-111 (¹¹¹In), iodine-131 (¹³¹I) rhenium-186 (¹⁸⁶Re,)and thallium-201 (²⁰¹Tl), terbium and their mixture thereof;
C) contrast-enhanced ultrasonography imaging compounds such as perfluoroctyl bromide (PFOB), acoustically active microbubbles and acoustically active liposomes;
D) fluorescence imaging compounds selected in the group consisting of quantum dots (i.e., fluorescent inorganic semiconductor nanocrystals), fluorescent dyes such as Texas red, fluorescein isothiocyanate (FITC), phycoerythrin (PE), rhodamine, fluorescein, carbocyanine, Cy-3™ and Cy-5™ (i.e., 3- and 5-N,N′-diethyltetra-methylindodicarbocyanine, respectively), Cy5.5, Cy7, DY-630, DY-635, DY-680 and Atto 565 dyes, merocyanine, styryl dye, oxonol dye, BODIPY dye (i.e., boron dipyrromethene difluoride fluorophore), and mixtures thereof;
E) X-ray contrast compounds such as iodine;
F) mixtures thereof.

In a second aspect, the invention relates to a method for preparing beads comprising radioactive imaging compounds comprising the following steps:
a) preparing an alkaline aqueous solution comprising an amount of fucoidan, an amount of at least one polysaccharide and an amount of a cross linking agent;
b) dispersing said alkaline aqueous solution into an hydrophobic phase comprising a surfactant in order to obtain w/o emulsion; and
c) transforming the w/o emulsion into beads by placing said w/o emulsion at a temperature from about 4° C. to about 80° C. for a sufficient time to allow the cross-linking of said amount of polysaccharide,
d) putting the obtained beads in contact with radioactive imaging compounds chosen in the group consisting of carbon 11 (¹¹C), nitrogen-13 (¹³N), oxygen-15 (¹⁵O) and fluorine-18 (¹⁸F), gallium-68 (⁶⁸Ga), yttrium-91 (⁹¹Y), indium-111 (¹¹¹In) rhenium-186 (¹⁸⁶Re), thallium-201 (²⁰¹Tl), terbium, and mixtures thereof,
   wherein
   said polysaccharide is selected from the group consisting of dextran, pullulan, fucoidan, agar, alginic acid, hyaluronic acid, inulin, heparin, chitosan and mixtures thereof.

In a specific embodiment, said polysaccharide is fucoidan. Step d) therefore provides a bead comprising fucoidan and at least one radioactive imaging.

In an alternative embodiment, the method of the invention comprises a step c′) after step c) and before step d) of incubating the beads obtained in step c) with a solution of fucoidan to graft fucoidan on the surface of the beads. In this embodiment, step d) provides a bead having fucoidan and at least one radioactive imaging compounds on its surface. In both of these embodiments, the bead of the invention comprises fucoidan and at least one radioactive imaging compound which allows the imaging entities of the invention to specifically target a given tissue, thanks to the selectivity of fucoidan toward selectins.

Typically, the step of dispersing the alkaline aqueous solution into the hydrophobic phase comprising a surfactant is performed under mechanical stirring. Typically, such a dispersing step is performed during 10 min. Alternatively, the emulsification process can be performed using a high performance disperser, such as Polytron® Homogenizer.

In a specific embodiment, the method of the invention further comprises the following steps:
v) or e) of submerging said polysaccharide beads into an aqueous solution; and
vi) or f) of washing said polysaccharide beads.

Typically, the beads are washed in water or phosphate buffer saline (PBS).

In another embodiment, the method of the invention further comprises a further step vii) or g) of calibrating the beads of the invention according to their size. Typically, the beads are calibrated according to their size using appropriate nylon filter. The skilled man is aware of the nylon filter adapted for the purpose of the invention.

In still another embodiment, the method of the invention further comprises a further step viii) or h) of freeze-drying said beads. Freeze-drying may be performed with any apparatus known in the art. There are essentially three categories of freeze dryers: rotary evaporators, manifold freeze dryers, and tray freeze dryers. Such apparatus are well known in the art and are commercially available such as a freeze-dryer Lyovac (GT2, STERIS Rotary vane pump, BOC EDWARDS). Basically, the vacuum of the chamber is from 0.1 mBar to about 6.5 mBar. The freeze-drying is performed for a sufficient time to remove at least 98.5% of the water, preferably at least 99% of the water, more preferably at least 99.5%. Typically, the freeze drying step is performed for 24 hours.

Preferably, the polysaccharide is a mixture of pullulan/dextran. Typically, the weight ratio of pullulan to dextran is 75:25 w/w.

Preferably, the weight ratio of pullulan to fucoidan is comprised between about 9:1 to about 9:2 w/w.

Typically, the imaging agent is in a solution or a suspension form. Preferably, the weight ratio of polysaccharides to imaging agent is comprised between about 50:1 to about 2:1. Typically, said cross-linking agent is selected from the group consisting of trisodium trimetaphosphate (STMP), phosphorus oxychloride (POCl₃), epichlorohydrin, formaldehydes, hydrosoluble carbodiimides, and glutaraldehydes. Preferably, for the purpose of the present invention, said cross-linking agent is STMP.

Typically, the weight ratio of the polysaccharide to the cross linking agent is in the range from 15:1 to 1:1, preferably 6:1.

The skilled artisan is aware of the hydrophobic phases suitable for the purpose of the present invention. Non-limiting examples of hydrophobic phases are vegetal oils, such as canola oil, corn oil, cottonseed oil, safflower oil, soybean oil, extra virgin olive oil, sunflower oil, palm oil, MCT oil, and trioleic oil. Preferably, for the purpose of the present invention, said hydrophobic phase is canola oil. Alternatively, said hydrophobic phase is a silicon fluid. Typically, the quantity of hydrophobic phase in the w/o emulsion (volume of lipophilic phase/volume of the water-in-oil emulsion; v/v) represents from 10% to 99% v/v, preferably from 20% to 80% v/v, preferably from 50% to 80% v/v and most preferably about 70% v/v of the w/o emulsion.

Typically, the surfactant present in the hydrophobic phase of step ii) or b) may be an ionic surfactant, such as sodium lauryl sulfate, or a, such as polyoxyethylene ethers, polyoxyethylene esters, and polyoxyethylene sorbitan and mixtures thereof. In a preferred embodiment, said surfactant is Tween 80.

In one embodiment of the invention, the alkaline aqueous solution of step i) or a) further comprises an amount of a porogen agent. Thus, the invention also provides porous beads. Non-limiting examples of porogen agents are sodium chloride, calcium chloride, ammonium carbonate, ammonium bicarbonate, calcium carbonate, sodium carbonate, and sodium bicarbonate and mixtures thereof. Preferably, for the purpose of the invention, said porogen agent is sodium chloride. Typically, the weight ratio of the polysaccharide to the porogen agent is in the range from 12:1 to 1:12. In a preferred embodiment, such weight ratio of the polysaccharide to the porogen agent is about 12:14. Typically, the density of the pores is from about 4% to 75%, preferably from about 4% to about 50%.

In a further embodiment, the alkaline solution further comprises a drug. The invention thus provides beads comprising a drug, said beads being highly adapted for administering said drug within a target tissue in the human or animal body. Typically, said drug is a drug having therapeutic effect, such as hormones, chemotactic agent, antibiotic, steroidal or non-steroidal analgesic, immunosuppressant, or anti-cancer drug.

In one particular embodiment, the method of the invention may comprise a further step consisting of hydrating the beads as prepared according to the invention. Said hydration may be performed by submerging the beads in a solution, preferably an aqueous solution (e.g., de-ionized water, water filtered via reverse osmosis, a saline solution, or an aqueous solution containing a suitable active ingredient) for an amount of time sufficient to produce a bead having the desired water content. Typically, when a bead comprising the maximum water content is desired, the bead is submerged in the aqueous solution for an amount of time sufficient to allow the bead to swell to its maximum size or volume. Typically, the bead is submerged in the aqueous solution for at least about 1 hour, preferably at least about 2 hours, and more preferably about 4 hours to about 24 hours. It is understood that the amount of time necessary to hydrate the bead to the desired level will depend upon several factors, such as the composition of the used polysaccharides, the size and thickness of the beads, and the temperature of the solution, as well as other factors.

The method of the invention can further include the step of sterilizing the beads using any suitable process. The beads can be sterilized at any suitable point. A suitable irradiative sterilization technique is for example an irradiation with Cesium 137, 35 Gray for 10 minutes. Suitable non-irradiative sterilization techniques include, but are not limited to, UV-exposure, gas plasma or ethylene oxide methods known in the art. For example, the beads can be sterilized using a sterilization system which is available from Abtox, Inc of Mundelein, Illinois under the trade mark PlazLyte, or in accordance with the gas plasma sterilization processes disclosed in U.S. Pat. No. 5,413,760 and U.S. Pat. No. 5,603,895.

In the specific embodiment, the beads of the invention comprise fucoidan. The presence of fucoidan is due to:
the presence of fucoidan in the alkaline aqueous solution of step i) or a) of the methods of the invention; and/or
the incubation of the beads of the invention with a solution of fucoidan during step iv) or c′) of the methods of the invention.

Fucoidans (also called fucosans or sulfated fucans) are sulfated polysaccharides with a wide spectrum of biological activities, including anticoagulant, antithrombotic, antivirus, antitumor, immunomodulatory, anti-inflammatory, and antioxidant activities (B. Li et al., Molecules, 2008, 13: 1671-1695; D. Logeart et al., J. Biomed. Mater Res., 1996, 30: 501-508). Fucoidans are α-1,2- or α-1,3- linked L-fucose polymers that are sulfated on position 4 and position 2 or 3 following the glycosidic linkage. However, besides fucose and sulfate residues, fucoidans also contain other monosaccharides (e.g., mannose, galactose, glucose, xylose, etc) and uronic acid groups. It is known in the art that the structure of fucoidans from different brown algae varies from species to species. Furthermore, the structure of fucoidans can also be chemically modified. For example, methods have been developed to increase the percentage of sulfate groups of fucoidans in order to obtain oversulfated fucoidans or fucoidan fragments (T. Nishino et al., Carbohydr. Res., 1992, 229: 355-362; S. Soeda et al., Thromb. Res., 1993, 72: 247-256).

In certain embodiments, the fucoidan moieties have an average molecular weight of about 2000 to about 9000 Da, e.g., about 5000, about 6000, about 7000 or about 8000 Da. In other embodiments, the fucoidan moieties have an average molecular weight of about 10,000 to about 90,000 Da, e.g., about 20,000, about 30,000, about 40,000, about 50,000, about 60,000, about 70,000 or about 80,000 Da. In yet other embodiments, the fucoidan moieties have an average molecular weight of about 100,000 to about 500,000 Da.

Fucoidan moieties suitable for use in the present invention are fucoidan moieties that have some degree of attraction for selectins and can play a targeting role when comprised in an imaging agent. Preferably, fucoidan moieties are stable, non-toxic entities that retain their affinity/specificity/selectivity properties under in vitro and in vivo conditions. In preferred embodiments, fucoidan moieties exhibit high affinity and specificity for selectins, i.e., they specifically and efficiently interact with, bind to, or associate with selectins. Suitable fucoidan moieties include fucoidans that exhibit affinity and specificity for only one of the selectins (i.e., for L-selectin, E-selectin or P-selectin) as well as fucoidans that exhibit affinity and specificity for more than one selectin, including those moieties which can efficiently interact with, bind to or associate with all three selectins. In certain embodiments, a suitable fucoidan moiety interacts with a selectin, preferably a human selectin, with a dissociation constant (K_{d}) between about 0.1 nM and about 500 nM, preferably between about 0.5 nM and about 10 nM, more preferably between about 1 nM and about 5 nM. The design of an inventive imaging agent will be dictated by its intended purpose(s) and the properties that are desirable in the particular context of its use. Thus, fucoidan moieties will be chosen based on their known, observed or expected, properties.

For example, in embodiments where the bead of the invention comprising fucoidan is to be used in the diagnosis of neurodegenerative disorders characterized by undesirable or abnormal selectin-mediated interactions in the brain, the bead will preferably be capable of crossing the blood-brain barrier. Therefore, such a bead will preferably contain a fucoidan moiety of low molecular weight (e.g., 2-8 kDa or lower than 5 kDa). In contrast, an imaging agent containing a fucoidan moiety of high molecular weight will be suited for situations in which the agent is to be used, for example to image selectins in the vascular system. Indeed, because of its high molecular weight, the bead will not be able to easily diffuse and will therefore more likely remain within the vascular system, thereby allowing a more selective targeting of the system of interest. A fucoidan moiety of high molecular weight can also have the advantage of being able to carry a high number of detectable moieties, thus increasing the sensibility of the imaging agent (i.e., allowing the detection of lower concentrations of selectins). In addition to their molecular weight, fucoidan moieties may be selected based on their sulfate content. By varying the sulfate content (either by selection of naturally-occurring fucoidans or by chemical modification), it may be possible to modulate the specificity of the fucoidan moiety (and corresponding imaging agent) for one of the selectins (L-selectin, E-selectin or P-selectin). It is known, for example, that binding to P- and E-selectins increases with the presence of sulfate groups on the ligand (T. V. Pochechueva et al., Bioorganic & Medicinal Chemistry Letters, 2003, 13: 1709-1712). Alternatively, a fucoidan moiety may be selected based on its structure and, in particular, based on the presence of at least one functional group that can be used (or that can be easily chemically converted to a different functional group that can be used) to associate the fucoidan moiety to a detectable compound. Examples of suitable functional groups include, but are not limited to, carboxy groups, thiols, amino groups (preferably primary amines), and the like.

Imaging agent are molecules that are detectable by imaging techniques such as ultrasonography, Magnetic Resonance Imaging (MRI), Positron Emission Tomography (PET), Single Photon Emission Computed Tomography (SPECT), fluorescence spectroscopy, Computed Tomography, X-ray radiography, or any combination of these techniques.

Preferably, imaging agent are stable and non-toxic.

In a first embodiment, said at least one imaging agent invention is an MRI imaging compound. In this embodiment, the bead of the invention is designed to be detectable by Magnetic Resonance Imaging (MRI). MRI, which is an application of Nuclear Magnetic Resonance (NMR), has evolved into one of the most powerful non-invasive techniques in diagnostic clinical medicine and biomedical research. It is widely used as a non-invasive diagnostic tool to identify potentially maleficent physiological anomalies, to observe blood flow or to determine the general status of the cardiovascular system. MRI has the advantage (over other high-quality imaging methods) of not relying on potentially harmful ionizing radiation.

Typically, said MRI imaging compound is a paramagnetic metal ion. Alternatively, said MRI Imaging compound is an ultrasmall superparamagnetic iron oxide (USPIO) particle. Therefore, said MRI imaging compound is selected in the group consisting of an ultrasmall superparamagnetic iron oxide (USPIO), gadolinium III (Gd³⁺), chromium III (Cr³⁺), dysprosium III (Dy³⁺), iron III (Fe³⁺), manganese II (Mn²⁺), and ytterbium III (Yb³⁺). In certain preferred embodiments, the paramagnetic metal ion is gadolinium III (Gd³⁺). Gadolinium is an FDA-approved contrast agent for MRI.

USPIO particles are composed of a crystalline iron oxide core containing thousands of iron atoms which provide a large disturbance of the Magnetic Resonance signal of surrounding water. In contrast to other types of nanoparticles such as quantum dots (currently under investigation as extremely sensitive fluorescent probes), USPIO particles exhibit a very good biocompatibility. Therefore, the beads of the invention comprising USPIO are highly appropriate use in imaging.

In a specific embodiment, the imaging agent is incorporated within a bead comprising fucoidan. In this embodiment, the bead of the invention is used for detecting selectins by MRI. Such imaging agents may be particularly useful in the diagnosis of cardiovascular pathologies associated with selectins. Indeed, with a diameter comprised between from 5 nm to 10 μm, preferably between 1 μm and 5 μm, beads of the invention are likely to diffuse only weakly outside the vascular space with the exception of more permeable pathological vascular tissues such as atherosclerotic walls.

The inventors have developed beads comprising a fucoidan and USPIO that proved to be efficient at detecting platelet-rich thrombus by MRI, with high sensitivity and specificity, when used for detecting an aneurysm of the abdominal aorta. Said beads showed a strong MRI contrast and a high affinity for the inner wall of an aneurysm.

In a second embodiment, said at least one imaging agent is a radioactive imaging compound. In this embodiment, the bead of the invention is designed to be detectable by a nuclear medicine imaging techniques such as planar scintigraphy (PS), Positron Emission Tomography (PET) and Single Photon Emission Computed Tomography (SPECT).

Preferably, said radioactive compound is selected in the group consisting of carbon-11 (¹¹C), nitrogen-13 (¹³N), oxygen-15 (¹⁵O) and fluorine-18 (¹⁸F), gallium-68 (⁶⁸Ga), yttrium-91 (⁹¹Y), technetium-99m (^{99m}Tc), indium-111 (¹¹¹In) iodine-131 (¹³¹I) rhenium-186 (¹⁸⁶Re), and thallium-201 (²⁰¹Tl), terbium and mixtures thereof.

SPECT and PET have been used to detect tumors, aneurysms, irregular or inadequate blood flow to various tissues, blood cell disorders, and inadequate functioning of organs, such as thyroid and pulmonary function deficiencies. Both techniques acquire information on the concentration of radionuclides introduced into a biological sample or a patient's body. PET generates images by detecting pairs of gamma rays emitted indirectly by a positron-emitting radionuclide. A PET analysis results in a series of thin slice images of the body over the region of interest (e.g., brain, breast, liver). These thin slice images can be assembled into a three dimensional representation of the examined area. However, there are only few PET centers because they must be located near a particle accelerator device that is required to produce the short-lived radioisotopes used in the technique. SPECT is similar to PET, but the radioactive substances used in SPECT have longer decay times than those used in PET and emit single instead of double gamma rays. Although SPECT images exhibit less sensitivity and are less detailed than PET images, the SPECT technique is much less expensive than PET and offers the advantage of not requiring the proximity of a particle accelerator. Planar scintigraphy (PS) is similar to SPECT in that it uses the same radionuclides. However, PS only generates 2D-information.

Thus, in certain embodiments, the at least one imaging agent is a radionuclide detectable by PET. Examples of such radionuclides include carbon-11 (¹¹C), nitrogen-13 (¹³N), oxygen-15 (¹⁵O) and fluorine-18 (¹⁸F). In other embodiments, the detectable compound is a radionuclide detectable by planar scintigraphy or SPECT. Examples of such radionuclides include technetium-99m (^{99m}Tc), gallium-68 (⁶⁸Ga), yttrium-91 (⁹¹Y), indium-111 (111_{In}), rhenium-186 (¹⁸⁶Re), thallium-201 (²⁰¹Tl) and terbium. Preferably, said imaging agent is technetium-99m (^{99m}Tc). Indeed, technetium-99m is highly appropriate since over 85% of the routine nuclear medicine procedures that are currently performed use radiopharmaceutical methodologies based on ^{99m}Tc.

The inventors have injected beads of the invention comprising a bead comprising fucoidan and ^{99m}Technietium in a healthy rat and a rat suffering suffering from an abdominal aortic aneurysm (AAA). They showed that radioactivity was found 4 times greater in the rat aorta suffering from AAA than that found in the rat aorta of healthy rat. Therefore they have shown that beads of the invention comprising a fucoidan and a radioactive imaging compound are accumulated at the aneurysm and are thus highly appropriate for imaging such aneurysm. In addition, the method of the invention confers valuable properties to the beads obtained thereof, enhancing their effectiveness as a tracer for scintigraphy since a larger number of imaging agent is grouped within the very structure of the beads which ultimately brings a much higher density of imaging agent at the specific site to be imaged. In addition, the microscopic size of the beads allows a better distribution of the imaging agent within the body. Finally, the beads being made of biodegradable polysaccharides, they do not constitute any danger for the subject in which the imaging techniques are operated.

In a third embodiment, said at least one imaging agent is a contrast-enhanced ultrasonography imaging compound. In this embodiment, the bead of the invention is designed to be detectable by contrast-enhanced ultrasonography (CEUS).

Ultrasound is a widespread technology for the screening and early detection of human diseases. It is less expensive than MRI or scintigraphy and safer than molecular imaging modalities such as radionuclide imaging because it does not involve radiation.

Preferably, said contrast-enhanced ultrasonography imaging compound is perfluoroctyl bromide (PFOB).

In a preferred embodiment, for carrying out this particular embodiment of the invention, the bead of the invention has a diameter comprised between 1 μm and 5 μm. Therefore, they are smaller than red blood cells, allowing them to flow easily through the circulation as well as the microcirculation (F. S. Vallanueva et al., Nat. Clin. Pract. Cardiovasc. Med., 2008, 5 Suppl. 2: S26-S32).

Preferably, the imaging agent comprises more than one molecule of PFOB. Consequently, they are highly valuable and overcome an important drawback of the imaging technique of prior art, i.e. they allow a better imaging (because of the numbers of molecules of PFOB within the imaging agent) and an enhanced distribution of the imaging agent within the body.

In a fourth embodiment, said at least one imaging agent is a fluorescence imaging compound. In this embodiment, the bead of the invention is designed to be detectable by fluorescence spectroscopy.

Favourable optical properties of fluorescent compound to be used in the practice of the present invention include high molecular absorption coefficient, high fluorescence quantum yield, and photostability. Preferred fluorescent moieties exhibit absorption and emission wavelengths in the visible (i.e., between 400 and 700 nm) or the near infra-red (i.e., between 700 and 950 nm). Selection of a particular fluorescent compound will be governed by the nature and characteristics of the illumination and detection systems used in the diagnostic method. In vivo fluorescence imaging uses a sensitive camera to detect fluorescence emission from fluorophores in whole-body living mammals. To overcome the photon attenuation in living tissue, fluorophores with emission in the near-infrared (NIR) region are generally preferred (J. Rao et al., Curr. Opin. Biotechnol., 2007, 18: 17-25). Numerous fluorescent compounds with a wide variety of structures and characteristics are suitable for use in the practice of the present invention. Preferably, said fluorescent imaging compound is selected in the group consisting of, quantum dots (i.e., fluorescent inorganic semiconductor nanocrystals) and fluorescent dyes such as Texas red, fluorescein isothiocyanate (FITC), phycoerythrin (PE), rhodamine, fluorescein, carbocyanine, Cy-3™ and Cy-5™ (i.e., 3- and 5-N,N′-diethyltetra-methylindodicarbocyanine, respectively), Cy5.5, Cy7, DY-630, DY-635, DY-680, and Atto 565 dyes, merocyanine, styryl dye, oxonol dye, BODIPY dye (i.e., boron dipyrromethene difluoride fluorophore), and mixtures thereof. More preferably, said fluorescent imaging compound is fluorescein isothiocyanate.

### Beads Obtainable According to the Invention

The invention relates to a new class of entities useful for imaging that comprise at least one imaging agent. Indeed, the invention relates to beads obtainable by the methods of the invention. These beads are the only ones which have the remarkable properties provided by the invention. They are valuable since they:
allow high contrast imaging;
exhibit low toxicity;
are biodegradable,
are ultimately metabolized and/or excreted by the subject, so that it limits any risk or contamination for said subject;
have an appropriate size for enhancing biodistribution;
permit free circulation within the patient's vascular system.

Preferably, said beads have a substantially spherical or ovoid shape.

Typically, said polysaccharide beads have a size comprised from 5 nm to 5 mm, preferably from 5 nm to 1 mm, preferably from 5 nm to 10 μm, preferably from 5 nm to 5 μm, more preferably from 1 μm to 5 μm. The size of the beads would be chosen with precaution by the skilled man in regard with the desired use. The size of the beads of the invention is dependent on the characteristics and parameters of the method of preparing such beads. Typically, the size of the beads of the invention may depend on the nature of the polysaccharide, the agitation provided during the process and the distribution of the polysaccharide within the beads.

The inventors showed that the beads of the invention comprising fucoidan are characterised by the presence of sulphur at their surface, whereas the beads not comprising fucoidan are characterised by the absence of sulphur at their surface.

This demonstrates the presence of fucoidan at the surface of beads comprising fucoidan.

Therefore, the presence of sulphur discriminates the beads of the invention and the beads of prior art.

The skilled person in the art may easily determine whether a given bead has sulphur on its surface or not. Typically, he may use confocal imaging techniques on beads prepared with fluorescent fucoidan. FITC-labelled fucoidan can be observed on the surface of beads as well as inside the bead structure.

In one particular embodiment, the beads of the invention comprise a drug. Said beads thus present the dual advantages to permit medical imaging while providing a drug for treating a patient.

The beads obtainable by the method of the invention can be packaged in any suitable packaging material. Desirably, the packaging material maintains the sterility of the beads until the packaging material is breached.

When used in the purpose of carrying out a scintigraphy, the beads may be stocked as long as necessary before putting them in contact with a radioactive imaging compound in order to obtain a polysaccharide bead comprising fucoidan and said radioactive imaging compound.

### Use of the Crosslinked Polysaccharide Beads Comprising an Imaging Agent According to the Invention

The invention relates to the use of the beads of the invention as contrasting agent.

More specifically, the invention provides beads obtainable by the methods of invention for use in Magnetic Resonance Imaging, wherein said at least one imaging agent is a MRI imaging compound. Preferably, said MRI imaging compound is selected in the group consisting of ultrasmall superparamagnetic iron oxide particles (USPIOs), gadolinium III (Gd³⁺), chromium III (Cr³⁺), dysprosium III (Dy³⁺), iron III (Fe³⁺), manganese II (Mn²⁺), and ytterbium III (Yb³⁺), and mixtures thereof. The invention further provides beads obtainable by the methods of invention for use in ultrasonography, wherein said at least one imaging agent is a contrast-enhanced ultrasonography imaging compound.
Preferably, said contrast-enhanced ultrasonography imaging compound is perfluoroctyl Bromide.

The invention also provides beads obtainable by the methods of the invention comprising fucoidan and at least one radioactive imaging compound for use in scintigraphy, wherein said radioactive imaging compound is selected in the group consisting of carbon-11 (¹¹C), nitrogen-13 (¹³N), oxygen-15 (¹⁵O) and fluorine-18 (¹⁸F), gallium-68 (⁶⁸Ga), yttrium-91 (⁹¹Y), technetium-99m (^{99m}Tc), indium-111 (¹¹¹In) iodine-131 (¹³¹I), rhenium-186 (¹⁸⁶Re), and thallium-201 (²⁰¹Tl), terbium and mixtures thereof.

In one aspect, the present invention provides beads obtainable according to the methods of the invention for use for detecting the presence of selectins in a patient, wherein said beads comprises fucoidan and an imaging agent. For this purpose, an effective amount of the imaging agent of the invention is administrated to the patient.

More specifically, the invention provides targeted reagents that are detectable by imaging techniques and methods allowing the detection, localization and/or quantification of selectins in in vitro and ex vivo systems as well as in living subjects, including human patients.

The present invention provides methods for detecting the presence of selectins in a biological system comprising the step of contacting the biological system with beads comprising an imaging agent obtainable by the method of the invention.

The contacting is preferably carried out under conditions that allow the imaging agent to interact with selectins present in the system so that the interaction results in the binding of the beads to the selectins thanks to the presence of fucoidan. The bead that is bound to selectins present in the system is then detected using an imaging technique. One or more images of at least part of the biological system may be generated. The contacting may be carried out by any suitable method known in the art. For example, the contacting may be carried out by incubation.

The biological system may be any biological entity that can produce and/or contain selectins. For example, the biological system may be a cell, a biological fluid or a biological tissue. The biological system may originate from a living subject (e.g., it may be obtained by drawing blood, by biopsy or during surgery) or a deceased subject (e.g., it may be obtained at autopsy). The subject may be human or another mammal. In certain preferred embodiments, the biological system originates from a patient suspected of having a clinical condition associated with selectins.

The administration is preferably carried out under conditions that allow the imaging agent (1) to reach the area(s) of the patient's body that may contain abnormal selectins (i.e., selectins associated with a clinical condition) and (2) to interact with such selectins so that the interaction results in the binding of the imaging agent to the selectins. After administration of the selectin-targeted imaging agent and after sufficient time has elapsed for the interaction to take place, the imaging agent bound to abnormal selectins present in the patient is detected by an imaging technique. One or more (e.g., a series) images of at least part of the body of the patient may be generated. The person skilled in the art will know, or will know how to determine, the most suitable moment in time to acquire images following administration of the imaging agent. Depending on the imaging technique used (e.g., MRI), one skilled in the art will also know, or know how to determine, the optimal image acquisition time (i.e., the period of time required to collect the image data).

Administration of the selectin-targeted imaging agent can be carried out using any suitable method known in the art such as administration by oral and parenteral methods, including intravenous, intraarterial, intrathecal, intradermal and intracavitory administrations, and enteral methods.

The beads comprising fucoidan and at least one imaging agent according to the methods of the invention are highly suitable for diagnosing a pathological condition associated with selectins. The diagnosis can be achieved by examining and imaging parts of or the whole body of the patient or by examining and imaging a biological system (such as one or more samples of biological fluid or biological tissue) obtained from the patient. One or the other method, or a combination of both, will be selected depending of the clinical condition suspected to affect the patient. Comparison of the results obtained from the patient with data from studies of clinically healthy individuals will allow determination and confirmation of the diagnosis.

The beads of the invention, used in medical imaging are also valuable for following the progression of a pathological condition associated with selectins. For example, this can be achieved by repeating the method over a period of time in order to establish a time course for the presence, localization, distribution, and quantification of “abnormal” selectins in a patient.

They can also be used to monitor the response of a patient to a treatment for a pathological condition associated with selectins. For example, an image of part of the patient's body that contains “abnormal” selectins (or an image of part of a biological system originating from the patient and containing “abnormal” selectins) is generated before and after submitting the patient to a treatment. Comparison of the “before” and “after” images allows the response of the patient to that particular treatment to be monitored.

Pathological conditions that may be diagnosed, or whose progression can be followed using the beads herein may be any disease and disorder known to be associated with selectins, i.e. any condition that is characterized by undesirable or abnormal interactions mediated by selectins. Examples of such conditions that may advantageously be diagnosed using methods provided herein include, but are not limited, thrombosis, myocardial ischemia/reperfusion injury, stroke and ischemic brain trauma, neurodegenerative disorders, tumor metastasis and tumor growth, and rheumatoid arthritis.In a specific embodiment, the beads of the invention may be detected within an organ or a tissue, preferably within a muscle. In this embodiment, the beads of the invention comprise a drug to be delivered in a said target organ or tissue. The presence of the beads is then determined by imaging techniques because of the presence of the imaging agent within the beads.

Therefore, the beads of the invention are suitable for delivering a drug while determining whether the beads have reached the targeted organ or tissue. The person skilled in the art would thus adapt the size of the beads suitable for this purpose, i.e. for targeting organs or tissues.

### FIGURES LEGEND

FIG. 1: Fluorescence confocal microscopy of large beads comprising FITC labeled fucoidan.

FIG. 2: Presentation of the composition of the large beads in percentage of signal detected by Energy-dispersive X-ray spectroscopy analysis for each element C, O, Na, Cl, Fe, F.

FIG. 3: Distribution of the size of the large beads according to the invention.

FIG. 4: Mean fluorescence intensity of small MP and MP fucoidan on platelet rich plasma (PRP) not activated, activated and activated then incubated 20 minutes with anti P-Selectin in order to block P-Selectin expression. The interaction of FITC fluorescent small MP and MP fucoidan are tested on unactivated platelets rich plasma (PRP), platetets rich plasma activated with TRAP (20 μM) and platelets rich plasma activated then P-Selectin blocked with CD62P (100 μM). The mean fluorescence intensity was measured in the area of double positivity.

FIG. 5: Quantification of the number of large beads comprising a fluorescent imaging agent in mice as a function of time after injection; large beads with and without fucoidan associated with the activated endothelium, control group (large beads with fucoidan associated with the non activated endothelium).

FIG. 6: Quantification of the number of large beads with fucoidan comprising an imaging agent (fluorescent FITC and/or echogenic PFOB and/or MPIO) versus control beads without fucoidan, associated with the activated endothelium (expressed per 100 leukocytes in the region of interest).

FIG. 7: MRI transversal T₂*-weighted images of the aneurysm of an AAA rat before injection and after injection into the carotid artery of large MPIO comprising fucoidan (200 μL of 150 mg/mL in 0.9% NaCl) at 65 minutes, 81 minutes and 115 minutes after injection.

FIG. 8: Ultrasound in two-dimensional mode of large beads of the invention (250 mg/mL in 0.9% NaCl) without PFOB (a) and with PFOB (b). Mean contrast (in dB) of large beads with and without PFOB (c)

FIG. 9: Ultrasound in two-dimensional mode of the aneurysm of an AAA rat before injection (a) and after injection into carotid artery of large microparticles comprising PFOB and fucoidan (200 μL of 150 mg/mL in 0.9% NaCl) at 5 seconds (b) and 5 minutes (c) after injection.

FIG. 10: Frontal section of scan images and superimposed (NanoSPECT/CTPLUS) after injection into the penis vein of small ^{99m}Tc-MP-fucoidan (200 μL of a 50 mg/mL suspension in NaCl 0.9%) in a healthy rat (a) and in a rat suffering from an AAA (c). Control after injection of non functionalized small ^{99m}Tc-MP in a rat suffering from an AAA (b).

FIG. 11: Average activity measured by autoradiography on sections of healthy rat abdominal aorta (n=17 slices) and rat suffering from AAA (n=35 slices). n=1 rat.

FIG. 12: Masson trichrome staining of an AAA cryosection from an AAA rat sacrificed 30 minutes after injection of small microparticles comprising fucoidan and FITC dextran (200 μL of a 50 mg/mL suspension in NaCl 0.9%). 3a. P-Selectin immunostaining (left) and control (right). 3b. Fluorescence microscopy. 3c. Alcian blue staining.

FIG. 13: Development and characterization of beads larger than 10 microns, observed by fluorescence microscopy
(a) The addition of PFOB in the preparation leads to the presence of non-fluorescent droplets, which correspond to the PFOB, observed within the MP
(b) Representative size distribution of the beads, prepared with or without PFOB, with an average size of 49 microns.
(c) The beads were observed by electron microscopy environmental
(d) Cavities were also observed in the MP prepared in the presence of PFOB
(e) The Energy-dispersive X-ray spectroscopy analysis confirmed the presence of fluorine in the beads MP-PFOB, whereas MP alone do not contain
(f). Ultrasound in two-dimensional mode of beads (250 mg/mL in 0.9% NaCl) without PFOB.
(g) Ultrasound in two-dimensional mode of beads (250 mg/mL in 0.9% NaCl) with PFOB

### EXAMPLES

### I—Development of Beads

### Reagents

Pullulan: 9 g (Hayashibara, M=200 000 g/mol);
Dextran: 3 g (Sigma, M=500 000 g/mol);
Dextran-FITC: 100 mg (Sigma, M=500 000 g/mol);
Fucoidan: 1.2 g (Sigma, M=57 000 g/mol);
Trisodium trimetaphosphate, STMP: 150 mg (Sigma, M=305.89 g/mol);
Rapeseed oil: 30 ml (Commercial, HLB=7);
Span 80: 7.5 g (Sigma, M=428.62 g/mol);
Tween 80: 3 g (Fluka Chemika, M=1310 g/mol); and
SnCl₂: 1 mg (Sigma, M=84 g/mol)

### Detectable Compounds

USPIO: 40 mL (Sinerem, Guerbet, 20 mg Fe/mL);
Perfluorooctyl Bromide, PFOB: 240 microL (Sigma, M=498.962 g/mol);
^{99m}technetium: 500 microL (corresponding to an activity of about 10 mCi; and Xavier Bichat Hospital, Department of Nuclear Medicine).

1. Preparation of Aqueous Phase

This aqueous phase is formed by pullulan and dextran, supplemented with one or more detectable compound (FITC fluorophore, USPIO, PFOB). This aqueous phase may be in the form of a solution, suspension or emulsion oil-in-water (O/W).

### Preparation of the Solution of Pullulan/Dextran

9 g of pullulan (Hayashibara, M=200,000 g/mol) and 3 g of dextran (Sigma, M=500,000 g/mol) are solubilised in 40 mL of purified water in a 100 mL beaker. The solution is stirred with a magnetic stirrer until obtaining a homogeneous solution.

### Preparation of the Solution Pullulan/FITC Dextran

9 g of pullulan (Hayashibara, 200,000 g/mol), 3 g of dextran (Sigma, 500,000 g/mol) and 100 mg of FITC-dextran (Sigma, 500,000 g/mol) are solubilised in 40 mL of purified water in a 100 mL beaker and the solution is stirred with a magnetic stirrer until obtaining an a homogeneous solution.

### Preparation of the Solution Pullulan/Dextran/Fucoidan

9 g of pullulan (Hayashibara, M=200,000 g/mol), 3 g of dextran (Sigma, M=500,000 g/mol) and 1.2 g of fucoidan (Sigma, M=57,000 g/mol) are solubilised in 40 mL of purified water in a 100 mL beaker and the solution is stirred with a magnetic stirrer until obtaining an homogeneous solution.

### Preparation of the Suspension of Pullulan/Dextran-USPIO

9 g of pullulan (Hayashibara, M=200,000 g/mol) and 3 g of dextran (Sigma, M=500,000 g/mol) are solubilised in 40 mL of a suspension of USPIO (Sinerem, Guerbet, 20 mg Fe/mL) in a 100 mL beaker and we stir with a magnetic stirrer until a homogeneous suspension.

### Preparation of the Solution Pullulan/Dextran/Fucoidan USPIO

9 g of pullulan (Hayashibara, M=200,000 g/mol), 3 g of dextran (Sigma, M=500,000 g/mol) and 1.2 g of fucoidan (Sigma, M=57,000 g/mol) are solubilised in 40 mL of a suspension of USPIO (Sinerem, Guerbet, 20 mg Fe/mL) in a 100 mL beaker and the solution is stirred with a magnetic stirrer until obtaining an homogeneous suspension.

### Preparation of Emulsion Pullulan/PFOB-Dextran

240 microL of PFOB (perfluorooctylbromide, Sigma, M=498.962 g/mol), 30 mg of Tween 80 (Sigma, M=428.62 g/mol) and 30 microL of NaOH (10M) were added to 300 mg of pullulan/dextran in a sample tube and we create an emulsion of PFOB in the pullulan/dextran by mixing with a dispenser (Polytron PT 3100, Kinematica) with a rod ( 5/7, 5 mm—REF: PT-07/2EC-B101 DA) for 20 seconds at 28 000 rev/min.

### Preparation of Emulsion Pullulan/Dextran/Fucoidan-PFOB

240 microL of PFOB (perfluorooctylbromide, Sigma, M=498.962 g/mol), 30 mg of Tween 80 (Sigma, M=428.62 g/mol) and 30 microL of NaOH (10M) were added to 300 mg of pullulan/Dextran-fucoidan in a sample tube and we create an emulsion of PFOB in the pullulan/Dextran-fucoidan by mixing with a dispenser (Polytron PT 3100, Kinematica) with a rod ( 5/7, 5 mm—REF 07/2EC-B101 PT-DA) for 20 seconds at 28 000 rev/min.

2. Preparation of Micro-Emulsification and Cross-Linking Process

### Preparation of the Oil Phase (Surfactant HLB 7)

7.5 g of Span 80 and 2.7 g of Tween 80 were mixed in a 10 mL beaker and the mixture was homogenised under magnetic stirring. 460 mg of this surfactant was then added to 30 mL of rapeseed oil in a bottle (bottle 30×70 PS, VWR Ref 216-2686), for a concentration of 1.5% (by volume) of surfactant. The solution is then put in a bottle bottle at −20° C. for 20 min in order to have a final oil phase at −5° C.

From this step, two experiments were performed. The first experiment resulted in suspensions of beads with diameters varying from 1 μm from 10 μm and a mean diameter from 1 to 2 μm. For sake of clarity, in the followings, the first experiment is referred to as the “large beads experiment”, whereas the second experiment is referred to as “the small beads experiment” The second experiment indeed resulted in suspensions of smaller beads, with diameters varying from 50 nm to 4 μm and a mean diameter from 300 nm to 600 nm.

Emulsification of the aqueous phase of polysaccharides in the oily phase 30 microL of NaOH (10M) was added to 300 mg of the aqueous phase for the large beads experiment, or to 100 mg of the aqueous phase for the small beads experiment. The resulting solution is mixed then incubated for 10 minutes at room temperature. In order to prepare beads comprising PFOB or beads comprising fucoidan and PFOB, there is no need to add NaOH. Indeed, the emulsions pullulan/dextran-PFOB or pullulan/dextran/fucoidan-PFOB already contain NaOH.

The rod in the bottle of oily phase is placed just above the level of the oil. 30 μl of a solution of the crosslinking agent STMP (Trisodium trimetaphosphate, Sigma, 305.89 g/mol) prepared in 30% (w/v) in water was added to the aqueous phase.

The resulting solution is stirred with the cone of the pipette. The aqueous phase was then collected with a pipette P5000. The aqueous phase is slowly injected to the oil phase under agitation provided with a disperser running at 28 000 rotations/min. The dispersion is performed until homogenisation.

The emulsion is then transferred in an oven, at a temperature of 50 C wherein the crosslinking step takes place for 20 minutes.

Said step results in crosslinked beads. Said beads are placed in PBS and are agitated for 40 minutes at room temperature. The oil phase is eliminated, and the beads are isolated.

In the large beads experiment, after centrifugation at 4100 rpm for 10 minutes, a pellet of beads is obtained. In the small beads experiment, after a first centrifugation at 4100 rpm for 10 minutes, the supernatant is taken carefully (avoiding that any bead from the pellet is taking with) and after a second centrifugation at 8000 rpm for 10 minutes, a pellet of beads is obtained. In both experiments, the supernatant is removed and PBS in added before mixing by vortexing the beads.

### Rinsing Beads

Centrifugation is performed again, at 4100 rpm for the large beads experiment or at 8000 rpm for the small beads experiment for 10 minutes, and the pellet of beads is resuspended in 0.04% SDS. This step is repeated two times.

After centrifugation at 4100 rpm/8000 rpm for 10 minutes, the pellet of beads is resuspended in 0.9% NaCl. This step is repeated six times.

### Filtration of Beads

In the large beads experiment, beads are sorted in solution in 0.9% NaCl through a sieve (AS 200, Retsch) combined with a filter paper nylon mesh opening 5 μm (SEFAR NITEX, 03-5/1 115 cm).

### Storage of Beads

After filtration, the suspension of beads in 0.9% NaCl was stored at 4° C.

3. Radiolabelling beads with Technetium (^{99m}Tc)

In order to obtain beads comprising ^{99m}Tc, the addition of ^{99m}Tc on beads is made in an extemporaneous manner.

For this purpose, 30 μl of a solution of SnCl₂ (1 mg/mL, Sigma, M=84 g/mol), were added to 500 μL of ^{99m}Tc₄⁻ and 500 μL of 0.9% NaCl to a pellet of 120 mg of beads in a 1.5 mL eppendorf.

Homogenisation is briefly performed with a vortex and the suspension is incubated 1 h at room temperature. The particles were then centrifuged and the supernatant was removed.

The labelling yield was calculated after measuring the radioactivity associated with particles and that found in the supernatant.

4. Grafting of Fucoidan on Polysaccharide Beads

The large beads of the invention (100 mg) were incubated with a solution of fucoidan (10 mg) in water (1 mL) followed by the addition of 10 μL NaOH 10M under magnetic stirring for 10 min. Grafting of fucoidan was performed by adding 3 mg of sodium trimetaphosphate (STMP) and by incubating the suspension for 20 min at 50° C. Beads were then washed by centrifugation 3 times with PBS and 3 times with purified water to remove any free reagents. When fluorescein-labeled fucoidan was grafted on rhodamine-labeled pullulan, fluorescence observations confirmed the presence of green fucoidan on red beads.

### II—Physicochemical Characterization of Beads

1. Form and Composition of Beads

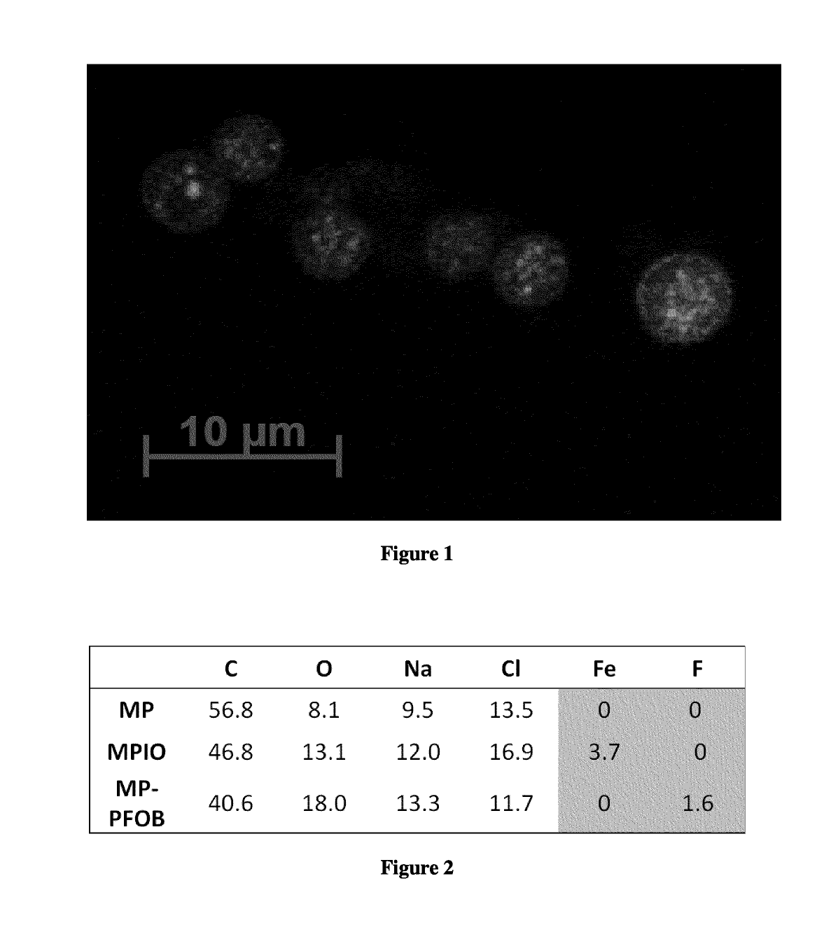

Energy-dispersive X-ray spectroscopy analysis on small and large beads suspension confirmed the presence of sulphur at the surface of the beads comprising fucoidan and the absence of sulphur at the surface of the beads not comprising fucoidan. This demonstrates the presence of fucoidan at the surface of beads comprising fucoidan. Additionally, the inventors performed confocal imaging on beads prepared with fluorescent fucoidan. FITC-labeled fucoidan was observed on the surface of beads and also inside the bead structure (FIG. 1)

Energy-dispersive X-ray spectroscopy analysis on sections of large beads of different formulations has allowed us to characterize their composition. The beads were included in blocks (50 μL of solutions at 250 mg/mL in 0.9% NaCl included in Cryomatrix™) frozen in contact with liquid nitrogen, then sectioned (8 microns) using a microtome and finally analyzed.

In each sample, the elements carbon and oxygen were found in large proportions (FIG. 2), which is quite normal since they are the key elements present in the polysaccharides used in the beads.

Significant amounts of sodium and chlorine were also found, the beads being suspended in 0.9% NaCl when analyzed. This basic analysis shows the presence of iron in the large beads comprising USPIO, and no iron in suspensions containing other types of large beads.

The presence of fluoride in suspensions of large beads comprising PFOB, while not in suspensions containing other types of large beads, demonstrates the presence of PFOB in the large beads comprising PFOB.

Atomic absorbtion spectroscopy measurement revealed that a suspension of large beads comprising USPIO (150 mg/mL in NaCl 0.9%) has an iron concentration of 11 mmol/L.

Gas chromatography—mass spectrometry analyses on a suspension of large beads comprising PFOB (150 mg/mL in NaCl 0.9%) indicated a concentration in PFOB of 8.47 mg/mL.

2. Diameter Size Distribution of Beads

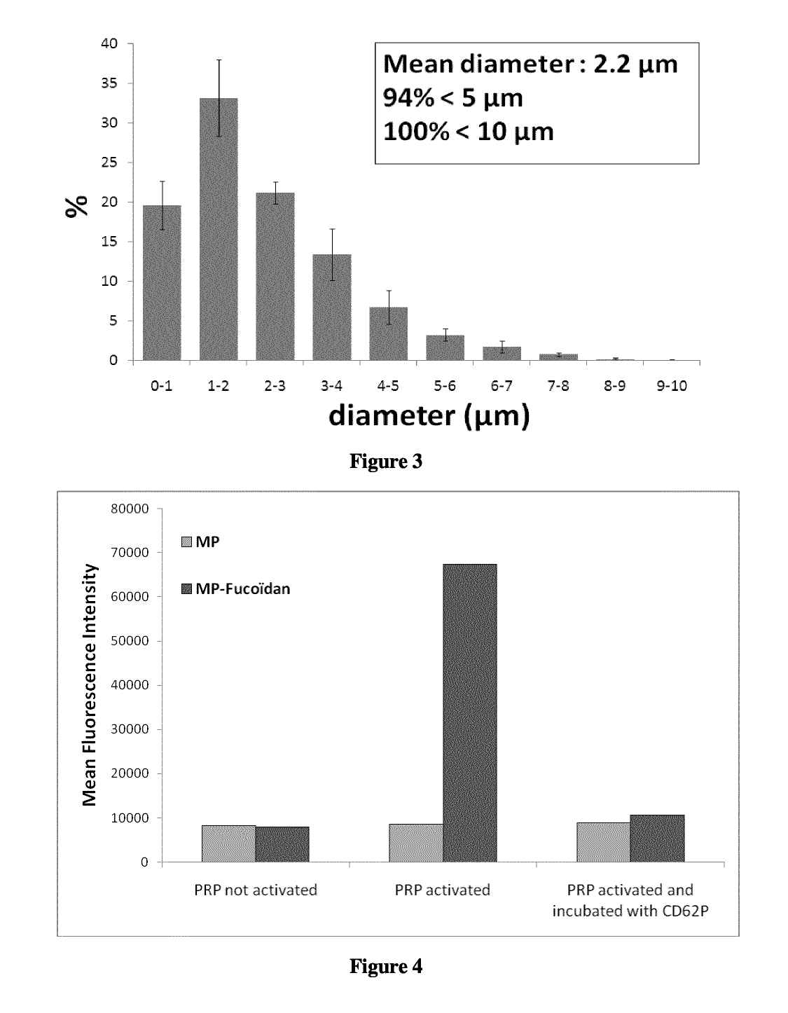

Large beads were prepared with dextran-FITC and were observed with optical fluorescence microscope. From digital photos, with the help of an image processing software, the size of the beads was measured. The distribution of particle size (in percentage) is shown in FIG. 3.

Small beads suspension was analyzed by dynamic light scattering method (Nano-ZS). A mean hydrodynamic diameter of 360 nm for beads not comprising fucoidan and 500 nm for small beads comprising fucoidan were found. Zeta potential were also measured and the beads comprising fucoidan had a higher electronegativity than the beads not comprising fucoidan (−16.2 mV vs −9.1 mV).

### III—Affinity of Beads Functionalized with Fucoidan

1. Affinity for Activated Platelets

In a first step, the inventors studied the in vitro interaction of small beads with activated platelets. Using flow cytometry, they showed the affinity of small beads functionalized with fucoidan for P-selectin expressed on the surface of activated platelets.

The beads are detected by green fluorescence (FITC) and platelets in red fluorescence (marking CD41-PE-Cy5). A double fluorescent element corresponds to the pair beads/platelets and the area of double positivity thus reflects the affinity between platelets and beads. The highest affinity (“Mean Fluorescence Intensity (MFI) of 67329”) is obtained with beads functionalized with fucoidan which were incubated with platelets activated with TRAP (20 μM) (FIG. 4).

The inventors noticed a weak affinity for these same beads when incubated with unactivated platelets (MFI of 7982) or with activated platelets incubated 20 minutes with anti P-Selectin in order to block P-Selectin expression (MFI of 10691). Finally, the inventors showed also a weak affinity for non-functionalized beads, whether they were incubated with activated, unactivated, or activated then blocked platelets (MFI of 8537, 8206 and 8833 respectively).

2. Affinity for Activated Endothelium

The inventors then assessed the affinity of the large beads for an activated endothelium. They used a model of inflammation of the calcium ionophore in the mouse mesentery. For this purpose, leukocytes were successfully labelled in red fluorescence by retro-orbital injection of rhodamine B (30 μl of a 0.3% solution). The inventors have then activated the endothelial wall by direct application of calcium ionophore (10 μl to 18 mM). Suspensions of beads or beads comprising fucoidan and FITC were then injected.

The inventors observed interactions at the activated site by intra-vital microscopy fluorescence.

An accumulation of leukocytes was observed at the area of interest, confirming the activation of the endothelium. A significant accumulation of large beads was also observed when they are functionalized with fucoidan. On the contrary, during an injection of non-functionalized beads, very few of them were found at the activated endothelial wall.

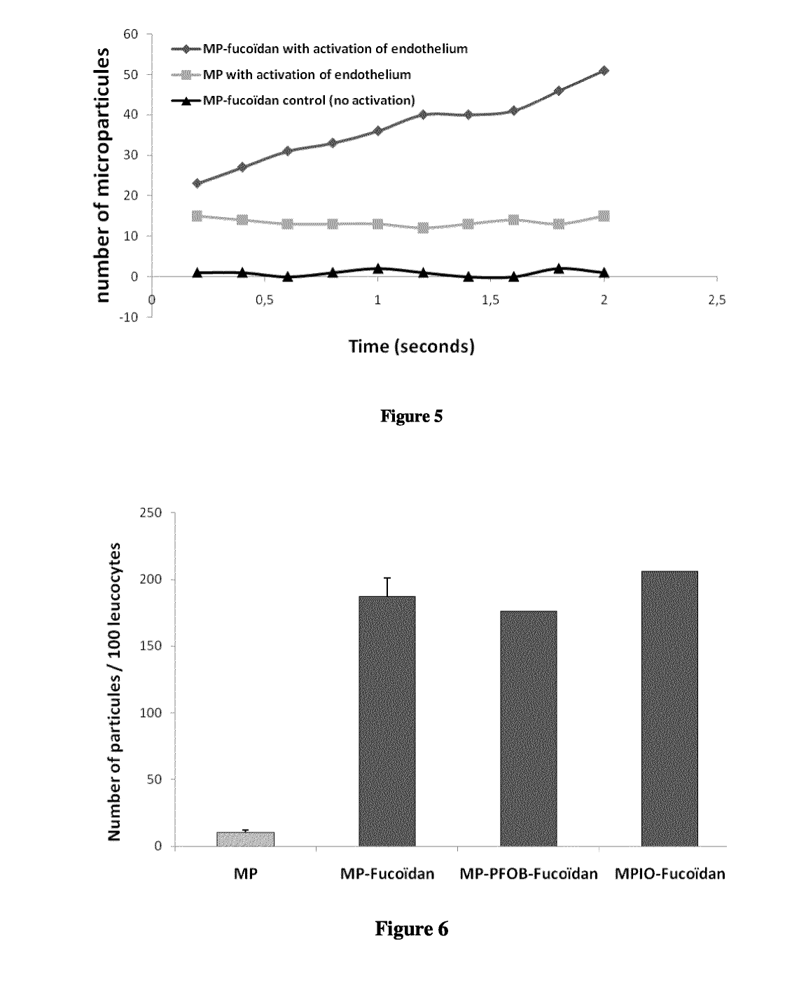

To characterize this high affinity, change in the number of beads found in the area of interest was measured. The inventors have thus shown that in the case of large beads prepared without fucoidan, the number of beads found in the activated endothelium is low and does not increase. On the contrary, in the case of injected large beads prepared with fucoidan, the number of beads comprising fucoidan found is higher and increases with time (FIG. 5).

The inventors further observed the lack of affinity for large beads comprising fucoidan for unactivated endothelium, since these particles are circulating and are not found at the area of interest. They also quantified the total number of beads accumulated at the activated endothelium at the end of the experiment, this number being expressed over the number of leukocytes found in the area of interest (FIG. 6).

The functionalized large beads have an affinity for an activated endothelium over 18 times greater than non-functionalized large beads (187 beads comprising fucoidan on average per 100 leukocytes versus 10 beads per 100 leukocytes). They further demonstrated that functionalized beads prepared in the presence of an imaging compound have a high affinity for activated endothelium, whatever the imaging agent encapsulated (206 beads with fucoidan and USPIO and 176 beads with fucoidan and PFOB, with results expressed per 100 leukocytes in the area of interest).

It is therefore possible to incorporate an imaging compound in the large beads without affecting their affinity for the activated endothelium. The inventors therefore have shown that the large beads of the invention prepared in the presence of fucoidan, with imaging agent or not, have a strong affinity for an activated endothelium.

### IV—Detection of Beads by MRI Imaging, Ultrasound and Scintigraphy

1. MRI

Ex vivo, the inventors tested the large MPIO by injecting them into an aneurysm of the abdominal aorta, which is a rat model developed in the laboratory.

After injection of the suspension of large MPIO, the inventors observed interactions by MRI. The beads comprising fucoidan and USPIO show a strong MRI contrast and a high affinity for the inner wall of an aneurysm.

The inventors then performed histological sections of the aneurysm in which functionalized MPIO were injected. For this purpose, the inventors performed an immunolabeling of P-selectin. They observed that the beads are preferentially localized at the wall of the aneurysm and the fragments of thrombus. In addition, areas where the beads are adsorbed on the wall correspond to areas where P-selectin is expressed and we find much iron in the same places that our beads.

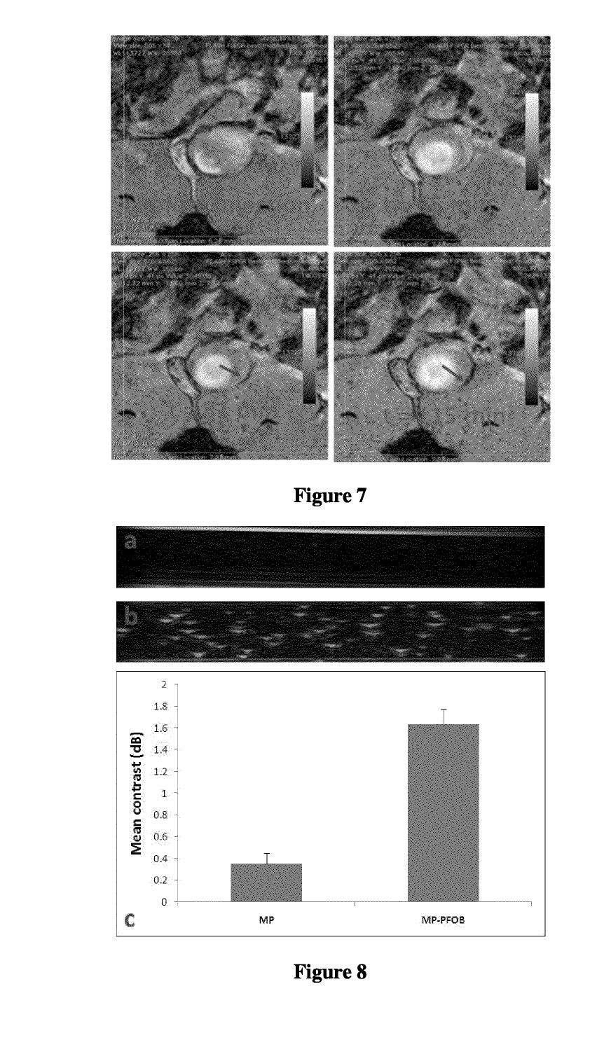

In vivo, the inventors injected the large MPIO comprising fucoidan, prepared with first example protocol, into the carotid artery of a rat suffering from an abdominal aortic aneurysm (AAA) and a strong MRI contrast were observed at the inner wall, 80 minutes after the injection (FIG. 7).

2. Ultrasound

In vitro, using a device for assessing the echogenic particle flow, the inventors demonstrated the echogenicity of the large beads PFOB of the invention (FIG. 8). They also showed the echogenicity of beads of average size 49 microns (FIG. 13).

Therefore, the inventors have demonstrated the echogenic characteristic of the PFOB large beads of the invention. Those results therefore corroborate that said beads are highly adapted for use as contrasting agents in vivo.

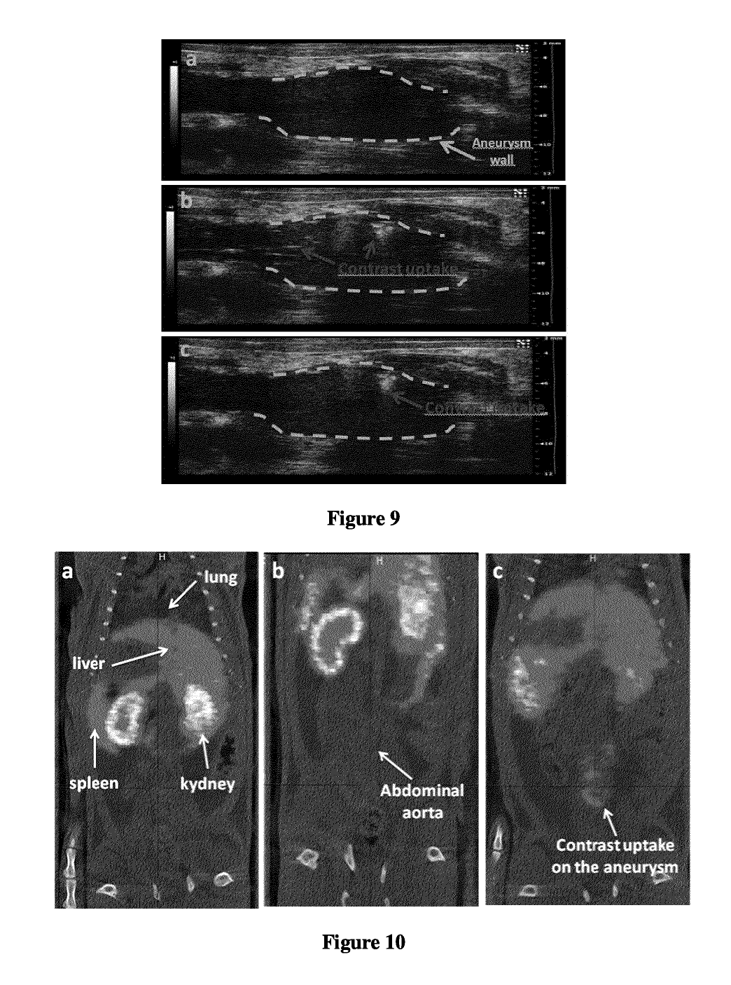

In vivo, the inventors tested the large beads comprising PFOB and fucoidan by injecting them (200 microL of 150 mg/mL beads in 0.9% NaCl) into the carotid artery of a rat AAA. Ultrasound imaging showed circulating echogenic beads in the abdominal aorta and accumulation of an echogenic signal in the aneurysmal area, 5 seconds after the injection (FIG. 9). As a control, the inventors tested the large beads comprising PFOB but not fucoidan and they showed that these circulating echogenic beads in the abdominal aorta did not accumulate in the aneurysmal area for up to 5 minutes after the injection.

3. Scintigraphy

To detect small and large beads by scintigraphy, the inventors have coupled them to ^{99m}technétium. The stability of this coupling in vitro in 0.9% NaCl was tested.

In plasma, the grafting of the technetium is considered stable for 1 hour. The inventors therefore studied the distribution of organic beads 30 minutes after the injection into the penis vein. The manipulation was performed after injection of small beads comprising or not fucoidan and radiolabeled with ^{99m}Tc in a healthy rat and in a rat AAA. Results are presented as percentage of radioactivity in each organ, compared to the total radioactivity injected. At the rat aorta suffering from abdominal aortic aneurysm, radioactivity was found 4 times greater than that found in the rat aorta of healthy rat (8.2% vs. 1.9%). The results indicate that the small beads of the invention are accumulated at the aneurysm.

The inventors further injected small beads comprising fucoidan and ^{99m}Tc (200 microL of 50 mg/mL beads in 0.9% NaCl) into the penis vein in order to image by in vivo scintigraphy the presence of these beads in the rat AAA (FIG. 10).

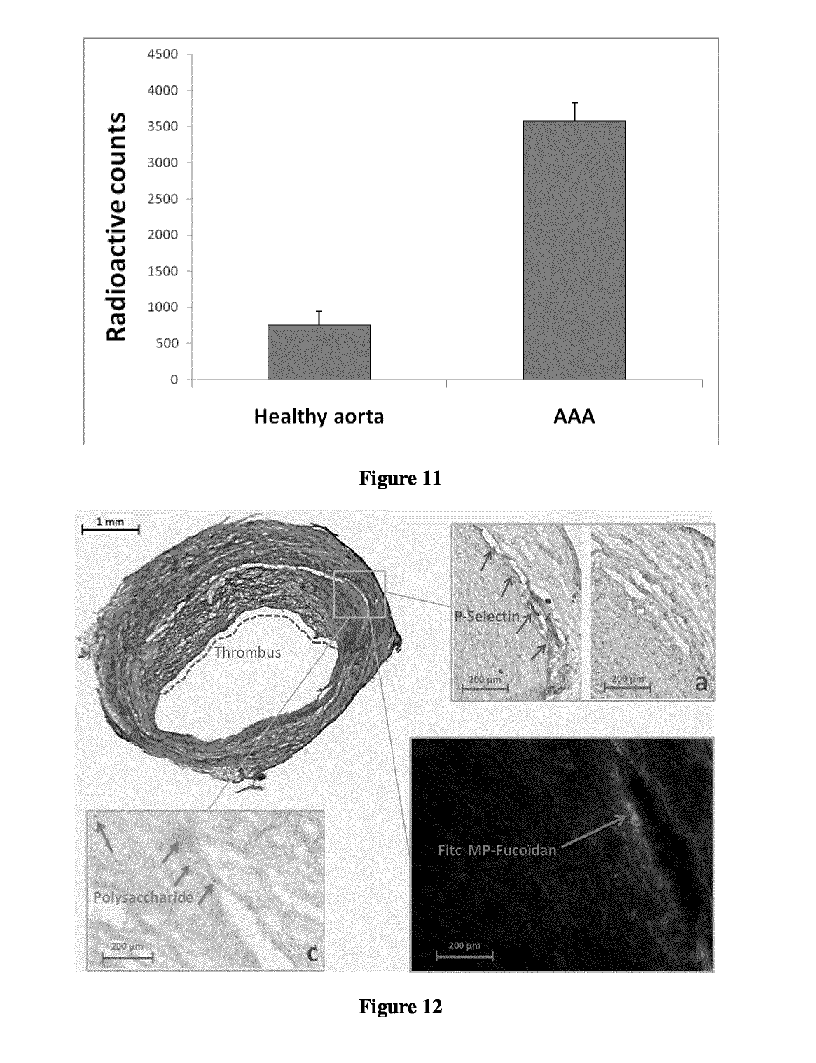

On the frontal section of a rat AAA, there is an obvious contrast enhancement in the aneurysm, compared with the abdominal aorta of a healthy rat and with a rat AAA injected with small beads comprising ^{99m}Tc but not fucoidan. These results indisputably show that small ^{99m}Tc-MP-fucoidan can be used to detect in vivo by scintigraphy, the presence of AAA in a rat. To quantify this signal, the inventors measured by autoradiography, the radioactivity found on activated cross-sections of 20 microns, produced by a microtome from abdominal aorta of healthy rats and rats bearing AAA (FIG. 11). The inventors find an average activity by cutting more than four times the level of the abdominal aorta of rat AAA compared with healthy cell of the rat (3575 counts versus 752 counts, respectively).

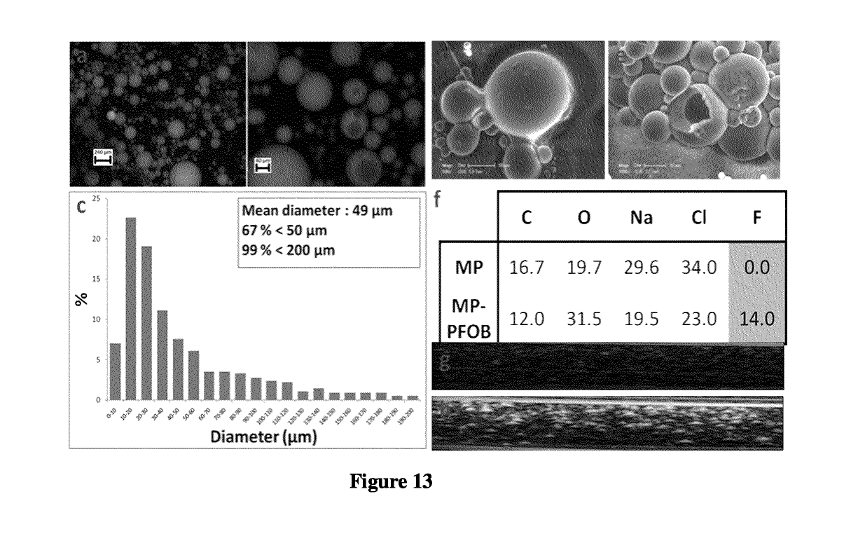

The inventors then performed histological sections of the aneurysm of the rat that was injected with functionalized radiolabeled small beads. For this purpose, the inventors performed an immunolabeling of P-selectin and a polysaccharide staining with alcian blue (FIG. 12). They observed that the beads are preferentially localized inside the wall of the aneurysm and the fragments of thrombus. In addition, areas where the beads are adsorbed inside the wall correspond to areas where P-selectin is expressed.

1. Method for preparing beads comprising an imaging agent comprising the following steps:
i) preparing an alkaline aqueous solution comprising an amount of at least one polysaccharide, an amount of an imaging agent and an amount of a cross linking agent;
ii) dispersing said alkaline aqueous solution into an hydrophobic phase comprising a surfactant in order to obtain w/o emulsion; and
iii) transforming the w/o emulsion into beads by placing said w/o emulsion at a temperature from about 4° C. to about 80° C. for a sufficient time to allow the cross-linking of said amount of polysaccharide:
wherein,
said polysaccharide is selected from the group consisting of dextran, pullulan, fucoidan, agar, alginic acid, hyaluronic acid, inulin, heparin, chitosan and mixtures thereof; and
wherein said at least one detectable compound is chosen among:
A) MRI imaging compounds selected in the group consisting of ultrasmall superparamagnetic iron oxide particles (USPIOs), gadolinium III (Gd³⁺), chromium III (Cr³⁺), dysprosium III (Dy³⁺), iron III (Fe³⁺), manganese II (Mn²⁺), and ytterbium III (Yb³⁺), and mixtures thereof;
B) radioactive imaging compounds selected in the group consisting of carbon-11 (¹¹C), nitrogen-13 (¹³N), oxygen-15 (¹⁵O) and fluorine-18 (¹⁸F), gallium-68 (⁶⁸Ga), yttrium-91 (⁹¹Y), technetium-99m (^{99m}Tc), indium-111 (¹¹¹In), iodine-131 (¹³¹I), rhenium-186 (¹⁸⁶Re), and thallium-201 (²⁰¹Tl), terbium and mixtures thereof;
C) contrast-enhanced ultrasonography imaging compounds, preferably perfluoroctyl bromide (PFOB), acoustically active microbubbles and acoustically active liposomes;
D) fluorescence imaging compounds selected in the group consisting of quantum dots, fluorescent dyes such as Texas red, fluorescein isothiocyanate (FITC), phycoerythrin (PE), rhodamine, fluorescein, carbocyanine, Cy-3, Cy-5, Cy5.5, Cy7, DY-630, DY-635, DY-680, and Atto 565 dyes, merocyanine, styryl dye, oxonol dye, BODIPY dye;
E) X-ray contrast compounds such as iodine; and
F) mixtures thereof.

2. The method according to claim 1, wherein said method further comprises a step iv) of incubating the beads obtained in step iii) with a solution of fucoidan to graft fucoidan on the surface of the beads.

3. The method according to claim 1, wherein said at least one detectable compound is an MRI imaging compound selected in the group consisting ultrasmall superparamagnetic iron oxide particles (USPIOs), gadolinium III (Gd³⁺), chromium III (Cr³⁺), dysprosium III (Dy³⁺), iron III (Fe³⁺), manganese II (Mn²⁺), and ytterbium III (Yb³⁺), and mixtures thereof.

4. The method according to claim 1 wherein said at least one detectable compound is a contrast-enhanced ultrasonography imaging compound, preferably such as perfluoroctyl bromide (PFOB).

5. Method for preparing beads comprising radioactive imaging compounds comprising the following steps:
a) preparing an alkaline aqueous solution comprising an amount of fucoidan, an amount of at least one polysaccharide and an amount of a cross linking agent;
b) dispersing said alkaline aqueous solution into a hydrophobic phase comprising a surfactant in order to obtain w/o emulsion;
c) transforming the w/o emulsion into beads by placing said w/o emulsion at a temperature from about 4° C. to about 80° C. for a sufficient time to allow the cross-linking of said amount of polysaccharide; and
d) putting the obtained beads in contact with radioactive imaging compounds chosen in the group consisting of carbon-11 (¹¹C), nitrogen-13 (¹³N), oxygen-15 (¹⁵O) and fluorine-18 (¹⁸F), gallium-68 (⁶⁸Ga), yttrium-91 (⁹¹Y), indium-111 (¹¹¹In), rhenium-186 (¹⁸⁶Re), thallium-201 (²⁰¹Tl), terbium, and mixtures thereof;
wherein said polysaccharide is selected from the group consisting of dextran, pullulan, fucoidan, agar, alginic acid, hyaluronic acid, inulin, heparin, chitosan and mixtures thereof.

6. The method according to claim 5, wherein said method further comprises a step c′) after step c) and before step d) of incubating the beads obtained in step c) with a solution of fucoidan to graft fucoidan on the surface of the beads.

7. The method according to claim 1, wherein said method further comprises the following steps:
v) or e) submerging said beads into an aqueous solution; and
vi) or f) washing said beads.

8. The method according to claim 1, wherein said cross-linking agent is selected from the group consisting of trisodium trimetaphosphate (STMP), phosphorus oxychloride (POCl₃), epichlorohydrin, formaldehydes, hydrosoluble carbodiimides, and glutaraldehydes.

9. The method according to claim 1, wherein said hydrophobic phase is selected from the group vegetal oils, preferably from canola oil, corn oil, cottonseed oil, safflower oil, soybean oil, extra virgin olive oil, sunflower oil, palm oil, MCT oil, and trioleic oil.

10. The method according to claim 1, wherein said at least one imaging agent is detectable by planar scintigraphy (PS), Single Photon Emission Computed Tomography (SPECT), Positron Emission Tomography (PET), contrast-enhanced ultrasonography (CEUS), Magnetic Resonance Imaging (MRI), fluorescence spectroscopy, Computed Tomography, ultrasonography, X-ray radiography, or any combination thereof.

11. A bead by the method of claim 1.

12. The bead according to claim 11, wherein said bead has a size comprised from 5 nm to 10 μm, preferably from 5 nm to 5 μm, more preferably from 1 μm to 5 μm.

13. A contrasting agent comprising the bead according to claim 11.

14. A magnetic resonance imaging agent comprising a Microparticle obtained by the method according to claim 3.

15. An ultrasonography agent comprising a Microparticle obtained by the method according to claim 4.

16. A scintigraphy agent compsiting a Microparticle obtained by the method according to claim 5.

17. An x-ray-based imaging agent comprising a Microparticle obtained by the method according to claim 1.

18. The method according to claim 5 wherein said method further comprises the following steps:
v) or e) submerging said beads into an aqueous solution; and
vi) or f) washing said beads.

19. The method according to claim 18, wherein said cross-linking agent is selected from the group consisting of trisodium trimetaphosphate (STMP), phosphorus oxychloride (POCl₃), epichlorohydrin, formaldehydes, hydrosoluble carbodiimides, and glutaraldehydes.

20. The method according to claim 5, wherein said hydrophobic phase is selected from the group vegetal oils, preferably from canola oil, corn oil, cottonseed oil, safflower oil, soybean oil, extra virgin olive oil, sunflower oil, palm oil, MCT oil, and trioleic oil.

21. A scintigraphy agent compsiting a Microparticle obtained by the method according to claim 6.
